(19) United States
(12) Patent Application Publication
(10) Pub. No.: US 2023/0404460 A1
(43) Pub. Date: Dec. 21, 2023

(54) ECG Monitoring Device Combined with BCG Signal

(71) Applicant: SHENZHEN GLAMOUR TECHNOLOGY CO., LTD, Shenzhen (CN)

(72) Inventors: Zhong LIU, Shenzhen (CN); Kuntao LU, Shenzhen (CN); Chengjun SUN, Shenzhen (CN); Yong LE, Shenzhen (CN)

(21) Appl. No.: 18/037,566

(22) PCT Filed: Dec. 21, 2020
(86) PCT No.: PCT/CN2020/138077

§ 371 Date: May 18, 2023

(30) Foreign Application Priority Data
Nov. 18, 2020 (CN) 202011298885.X

(51) Int. Cl. A61B 5/346; A61B 5/11; A61B 5/00

(52) U.S. Cl. CPC A61B 5/346; A61B 5/746; A61B 5/1102

(57) ABSTRACT

A dual-signal collection electrode sticker for an ECG monitoring device, comprising an ECG signal collection electrode for acquiring ECG signals and a BCG signal collection sensor for acquiring BCG signal. The ECG signal collection electrode is a single electrode, and the BCG signal collection sensor is dual electrodes.

### CROSS REFERENCE

The present application is a continuation of International Application No. PCT/CN2020/138077, with an international filing date of Dec. 21, 2020, which is based upon and claims priority benefits to Chinese Patent Application No. 202011298885, filed on Nov. 18, 2020, the contents of which are incorporated herein by reference.

### TECHNICAL FIELD

The present application belongs to the field of medical equipment, and in particular relates to an ECG monitoring device combined with BCG signal.

### BACKGROUND

Health has become a growing concern in modern society, and vital signs signals is an important signal to judge the health status of the human body, which including the information of electrical activity related to heart and lung function. At present, the equipment used to monitor human vital signs is still the mainstream multi-parameter monitor. The multi-parameter monitor is a kind of medical equipment which is used for detecting many physiological parameters such as ECG signal, heart rate, oxygen saturation and blood pressure in real time. It can realize the monitoring and early warning of human vital signs and provide effective clinical diagnosis basis for medical staff. The working principle of the ECG monitoring function of the multi-parameter monitor or ECG monitor is to collect weak ECG (Electrocardiogram) signals on the body surface of the human body through electrodes, and the ECG signal are the main signals sources for assisting in judging whether there are vital signs in the human body.

Under normal circumstances, ECG records the changes in electrical activity generated by each cardiac cycle of the heart, reflecting whether the heart is working normally, but for some special cases, such as cardiac electromechanical dissociation, the monitor still has ECG signal, and the ECG cannot reflect the abnormality of the heart. Cardiac electromechanical dissociation refers to the fact that the heart has electrical activity while without effective mechanical (pump) action, which is often a manifestation of dying and a form of sudden death. Simply put, the heart has stopped pumping blood, and the monitor still shows ECG signal, which can easily lead to misjudgment of vital signs of the human body, and once this happens, it will be fatal to the patient.

Therefore, it is necessary to design an optimized ECG monitoring device for the situations of above abnormal cardiac work, such as cardiac electromechanical dissociation.

### SUMMARY

Embodiments of the present invention aim to provide an ECG monitoring device that combines with BCG (Ballistocardiogram) signal, and aims to solve the problem that ECG signal in ECG monitors in the related art cannot accurately reflect abnormal working of the heart.

The embodiment of the present invention is realized as follows: a dual-signal collection electrode sticker for ECG monitoring device is provided, wherein the dual-signal collection electrode sticker comprises an ECG signal collection electrode for collecting ECG signal and a BCG signal collection sensor for collecting BCG signal, the ECG signal collection electrode is a single electrode, the BCG signal collection sensor is dual electrodes, the dual-signal collection electrode sticker is connected with a wired cable, and the other end of the wired cable is connected with a host, which is used for transmitting the ECG signal and the BCG signal to the host; when the ECG monitoring device monitors that the ECG signal exists and the BCG signal is abnormal, the abnormal activity of the heart is determined.

Further, the BCG signal collection sensor converts the vibration signals into an electrical signal, and the sensing material thereof is a piezoelectric material or a piezoresistive material.

Further, the BCG signal collection sensor is a piezoelectric type sensor, such as a piezoelectric film sensor, a piezoelectric cable sensor, or a piezoelectric ceramic sensor.

Further, the dual-signal collection electrode sticker comprises an electrode cable interface, a BCG sensing layer and an ECG electrode layer comprising conductive gel, wherein the electrode cable interface is located at the center of the dual-signal collection electrode sticker, and the BCG sensing layer is located above the ECG electrode layer.

Further, the BCG sensing layer comprises two layers of BCG sensing electrodes and a layer of BCG piezoelectric sensing material positioned between the two layers of BCG sensing electrodes, the electrode cable interface comprises two BCG cables and one ECG cable, the two BCG cables are respectively connected with the two layers of BCG sensing electrodes, and the ECG cable is connected with the ECG electrode layer.

Further, the dual-signal collection electrode sticker is in a flat cake-shaped structure as a whole.

Further, the ECG signal collecting electrode collects the ECG signal in the manner of ECG lead.

In another aspect, the embodiment of the present invention provides an ECG monitoring device combined with BCG signal, which comprises the above dual-signal collection electrode sticker, a wired cable and a host.

Further, the host comprises an ECG signal analysis unit, a BCG signal analysis unit, an alarm logic unit and an alarm, wherein the ECG signal analysis unit is used for processing the ECG signal to obtain an analysis result; the BCG signal analysis unit processes the BCG signal to obtain an analysis result; the alarm logic unit is used for determining whether a vital sign signals, an ECG signal or a BCG signal is abnormal in a human body, and sending an alarm control signals to the alarm under the condition that the vital signs signals of the human body are lost, the ECG signal is abnormal or the BCG signal is abnormal; and the alarm emits a warning alarm after receiving the alarm control signal.

Further, when the analysis result of the ECG signal analysis unit is abnormal and the analysis result of the BCG signal analysis unit is normal, the alarm logic unit determines that the ECG signal of the human body is abnormal; when the analysis result of the ECG signal analysis unit is normal and the analysis result of the BCG signal analysis unit is abnormal, the alarm logic unit determines that the BCG signal of the human body is abnormal; the alarm logic unit determines that the vital signs signals of the human body are lost when both the analysis result of the ECG signal analysis unit and the analysis result of the BCG signal analysis unit is abnormal.

Compared with the related art, the ECG monitoring device combined with the BCG signal provided by the present invention has the following beneficial effects: in that related art, on the basis of only collecting ECG signal through electrodes or electrode stickers, the invention increases the collection of BCG signal, the BCG signal reflects the blood pumping function of the heart, when the heart is abnormal and still has ECG signals, such as under the condition of cardiac electromechanical dissociation, the BCG signal is also abnormal, the abnormal BCG signal can reflect the abnormal activity of the heart, therefore, the defect that the ECG signal cannot accurately reflect the abnormal activity of the heart is made up to a certain extent. The ECG monitoring function of the multi-parameter monitor or the electrocardiographic monitor in the related art is improved, the medical staff can be assisted to judge the vital signs of the testee more timely and accurately, and the sudden death caused by the failure to find the change of the vital signs of the testee in time can be prevented to a certain extent.

### BRIEF DESCRIPTION OF THE DRAWINGS

In order to illustrate the technical solutions more clearly in the embodiments of the present invention or the related art, the attached drawings needed to be used in the description of the embodiments or the related art will be briefly introduced below. Obviously, the attached drawings in the following description are only some embodiments of the present invention, and a person of ordinary skill in the art may also derive other drawings according to these attached drawings without creative work.

FIG. 1 is a schematic structural diagram of an ECG monitoring device combined with BCG signal according to an embodiment of the present invention;

FIG. 2 is a schematic structural diagram of a dual-signal collection electrode sticker in an ECG monitoring device combined with BCG signal according to an embodiment of the present invention;

FIG. 3 is a schematic diagram of an internal structure of a dual-signal collection electrode sticker in an ECG monitoring device combined with BCG signal according to an embodiment of the present invention.

### DETAILED DESCRIPTION OF THE EMBODIMENTS

In order to make the technical problems to be solved, technical solutions and beneficial effects of the present invention clear, the present invention is further described in detail below with reference to the attached drawings and embodiments. It should be understood that the specific embodiments described herein are only used to explain the present invention, and are not used to limit the present invention.

In the following description, words such as “module”, “part”, or “unit” for indicating elements are used only for the convenience of explanation of the present invention, and are not intended to be particularly limiting, and “module” and “unit” may be mixed.

Ballistocardiogram (BCG) technique that is cited in the embodiment of the invention is a non-invasive technique that accurately describes the physical activity caused by the impact force generated by heart contraction and blood ejection and deceleration of blood flow through large blood vessels through a graphical interface. BCG itself is a signal of mechanical vibration. Every time the heart beats, blood is ejected from the heart into the blood vessels, and in the process of this ejection, there is a very slight force on the body. It can indirectly reflect the working state of the heart by detecting the weak vibration transmitted from the heart beat to the body surface, which is an advanced detection method for evaluating the dynamic performance of the human heart without feeling.

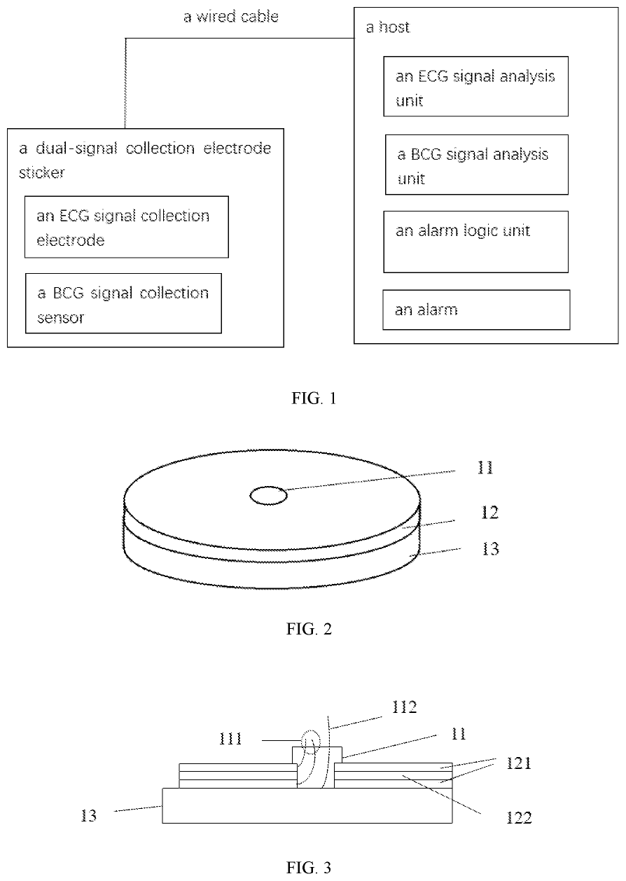

As shown in FIG. 1, an embodiment of the present invention provides an ECG monitoring device combined with BCG signal, which is used for collecting and monitoring ECG signal and BCG signal of a human body, comprising: a dual-signal collection electrode sticker, a wired cable and a host. The dual-signal collection electrode sticker comprises an ECG signal collection electrode and a BCG signal collection sensor. The ECG signal collection electrode is used for collecting an ECG signal (electrocardiographic signal), the BCG signal collection sensor is used for collecting a BCG signal (ballistocardiograph signal), and the ECG signal collection electrode is a single electrode, and the BCG signal collection sensor is dual electrodes.

Unlike an ECG signal, which is an electrical signal, the BCG signal is a mechanical force signal that is a periodic micro-vibration signals on the body due to the heart pumping blood. In the embodiment of the invention, on the basis that the ECG signal is collected only through the electrode or the electrode sticker in the related art, the collection of the BCG signal is added. Because the BCG signal reflects the blood pumping function of the heart, when the heart is abnormal and the ECG signals still exists, such as under the situation of cardiac electromechanical dissociation, the BCG signal is also abnormal, and the abnormal BCG signal can reflect the abnormal activity of the heart, so that the defect that a single ECG signal is insufficient to reflect the abnormal activity of the heart is made up to a certain extent, and the ECG monitoring function of the multi-parameter monitor or the ECG monitor in the related art is improved. The ECG signal collecting electrode collects the electrical signals (i.e. ECG signal) on the body surface of the human body can in the same way as the existing monitor collects the ECG signal, that is, the ECG lead, which is achieving by placing the electrodes or electrode stickers on different parts of the human body, and connecting the lead wires to the positive and negative electrodes of the monitor ammeter. For example, the current international 12-lead system, including limb leads connected with limbs and chest leads connected with chest.

The ECG signal collecting electrode also comprises conductive gel, and the conductive gel is sticky, so that the ECG signal collecting electrode can be directly attached to the skin of a human body, which is convenient to collect ECG signal of each part of the body.

The BCG signal collection sensor is a sensor that converts a weak vibration signal into an electrical signal, and the sensing material of the sensor can be a piezoelectric material or a piezoresistive material.

In one embodiment of the present invention, the BCG signal collection sensor is a piezoelectric film sensor or a piezoelectric cable sensor. The piezoelectric film sensor collects BCG signal in the process of heart contraction, blood ejection and deceleration of blood flow passing through large blood vessels. The principle is that the contraction of heart, blood ejection and deceleration of blood flow passing through large blood vessels will transmit pressure to the piezoelectric film sensor, and the sensor will convert pressure signals into electrical signals, which is called BCG signal. In general, the BCG signal collection sensor does not need to use an electrode sticker to contact the body to collect the BCG signal, so that the heart activity can be monitored under the condition that the testee does not feel. For example, the piezoelectric film sensor can collect the BCG signal only by being placed at the position near the chest on the mattress corresponding to the human body, and the signals collection method is suitable for monitoring the heart activity of the testee in real time for 24 hours, and can also be used for monitoring when the testee lies on the bed, sleeps at night, and the sleep of the testee will not be affected.

The wired cable is simultaneously connected with the dual-signal collection electrode sticker and the host, and is used for transmitting the ECG signal and the BCG signal collected by the dual-signal collection electrode sticker to the host.

The host comprises an ECG signal analysis unit, a BCG signal analysis unit, an alarm logic unit and an alarm, and is used for analyzing the collected ECG signal and the BCG signal, and emits a warning alarm under the condition that the human body vital signs signals are lost or the ECG signal is abnormal or the BCG signal is abnormal. The ECG signal analysis unit is used for processing the collected ECG signal to obtain an analysis result, and determine that the analysis result is normal if the analysis result is within a range specified by standard ECG signal; otherwise, the analysis result is determined to be abnormal. The BCG signal analysis unit is used for processing the collected BCG signal to obtain an analysis result, and determine that the analysis result is normal if the analysis result is within a range specified by standard BCG signal; otherwise, the analysis result is determined to be abnormal. The alarm logic unit determines whether the vital signs signals of the human body are lost, the ECG signal is abnormal, or the BCG signal is abnormal according to the analysis result of the ECG signal analysis unit and the analysis result of the BCG signal analysis unit, and sends an alarm control signal to the alarm under the condition that the vital signs signals of the human body are lost, the ECG signal is abnormal, or the BCG signal is abnormal. After receiving the alarm control signals sent by the alarm logic unit, the alarm emits a warning alarm so as to timely remind the medical staff that the life of the patient may be in danger.

When the analysis result of the ECG signal analysis unit is abnormal and the analysis result of the BCG signal analysis unit is normal, the alarm logic unit determines that the current ECG signal of the human body is abnormal; when the analysis result of the ECG signal analysis unit is normal and the analysis result of the BCG signal analysis unit is abnormal, the alarm logic unit determines that the BCG signal of the current human body is abnormal; when the analysis result of the ECG signal analysis unit and the analysis result of the BCG signal analysis unit are both abnormal, the alarm logic unit determines that the current vital signs signals of the human body are lost.

When the analysis result of the ECG signal analysis unit is normal and the analysis result of the BCG signal analysis unit is abnormal, or when the analysis result of the ECG signal analysis unit and the analysis result of the BCG signal analysis unit are both abnormal, that is, when the ECG monitoring device detects that the ECG signal exists and the BCG signal is abnormal, the alarm logic unit sends an alarm control signals to the alarm, and the alarm gives an alarm after receiving the alarm control signals, so that medical staff can timely find the changing situation of the vital sign of the patient, and the electrocardiographic monitoring device can timely and accurately assist the medical staff to determine the vital sign of the patient when the cardiac electromechanical dissociation occurs, and the amount of sudden death of the patient caused by the failure to timely find the changing situation of the vital sign of the patient can be reduced.

FIG. 2 and FIG. 3 show a structural diagram and an internal structural diagram of a dual-signal collection electrode sticker in an embodiment of the present invention. As shown in FIG. 2, the dual-signal collection electrode sticker comprises an electrode cable interface 11, a BCG sensing layer 12 and an ECG electrode layer 13 comprising conductive gel. The BCG sensing layer 12 is configured to collect BCG signal, the ECG electrode layer 13 is configured to collect ECG signal, and the electrode cable interface 11 is configured to transmit the collected BCG signal and ECG signal. The whole dual-signal collection electrode sticker is in a flat cake structure with a radius of 6 mm-20 mm, the electrode cable interface 11 is a round hole or an opening, and is located at the center of the dual-signal collection electrode sticker; the BCG sensing layer 12 is located above the ECG electrode layer 13, and the bottom of the ECG electrode layer 13 is made of conductive gel, which is sticky, so that the dual-signal collection electrode sticker can be directly attached to the skin of a human body, and is convenient to use. Furtherly, referring to FIG. 3, the BCG sensing layer 12 includes two layers of BCG sensing electrodes 121 and a layer of BCG piezoelectric sensing material 122 located between the two layers of BCG sensing electrodes 121. The electrode cable interface 11 comprises two BCG cables 111 and an ECG cable 112, the two BCG cables 111 are connected to the two BCG sensing electrodes 121 of the BCG sensing layer for transmitting the collected BCG signal, and the ECG cable 112 is connected to the ECG electrode layer 13 for transmitting the collected ECG signal.

Different from the traditional electrode sticker which can only collect the electrical signals of the human body, the dual-signal collection electrode sticker provided by the embodiment of the present invention integrates two BCG sensing electrodes and one ECG electrode into one electrode sticker, and on the basis of the function of collecting the ECG signal, the dual-signal collection electrode sticker can also collect the BCG signal reflecting the blood pumping function of the heart, thereby making up for the defect that the ECG signal cannot accurately reflect the abnormal activities of the heart, and improving the monitoring function of the electrode sticker on the changes of the vital signs of the human body.

The dual-signal collection electrode sticker can be a conventional electrode sticker, and the whole electrode sticker is round, square or other shapes.

In addition, the functional units in the embodiments of the present invention may be integrated into one processing unit, or may be physically independent of each other, or two or more units may be integrated into one unit. The integrated unit may be implemented in the form of hardware or in the form of a software program module.

The above description is only preferred embodiments of the present invention, and is not intended to limit the present invention. Any modification, equivalent substitution, and improvement made within the principle of the present invention shall be included in the protection scope of the present invention.

What is claimed is:

1. A dual-signal collection electrode sticker, wherein the dual-signal collection electrode sticker comprises an ECG signal collection electrode for collecting ECG signal and a BCG signal collection sensor for collecting BCG signals, and the ECG signal collection electrode is a single electrode and the BCG signal collection sensor is dual electrodes; the BCG signal collection sensor converts vibration signals into electrical signals, and a sensing material thereof is a piezoelectric material or a piezoresistive material.

2. The dual-signal collection electrode sticker of claim 1, wherein the BCG signal collection sensor is a piezoelectric type sensor.

3. The dual-signal collection electrode sticker of claim 2, wherein the piezoelectric type sensor is a piezoelectric film sensor, or a piezoelectric cable sensor, or a piezoelectric ceramic sensor.

4. The dual-signal collection electrode sticker of claim 1, wherein the dual-signal collection electrode sticker comprises an electrode cable interface, a BCG sensing layer and an ECG electrode layer comprising a conductive gel, the BCG sensing layer being located above the ECG electrode layer.

5. The dual-signal collection electrode sticker of claim 4, wherein the BCG sensing layer comprises two layers of BCG sensing electrodes and a layer of BCG piezoelectric sensing material positioned between the two layers of BCG sensing electrodes, the electrode cable interface comprising two BCG cable wires and an ECG cable wire, the two BCG cable wires being respectively connected with the two layers of BCG sensing electrodes, the ECG cable wire is connected with the ECG electrode layer.

6. The dual-signal collection electrode sticker of claim 1, wherein the dual-signal collection electrode sticker is in a flat cake-shaped structure as a whole.

7. An ECG monitoring device comprising a dual-signal collection electrode sticker of claim 1, further comprising a wired cable and a host.

8. The ECG monitoring device of claim 7, wherein the host comprises an ECG signal analysis unit, a BCG signal analysis unit, an alarm logic unit and an alarm, the ECG signal analysis unit is used for processing the ECG signal to obtain analysis results; the BCG signal analysis unit processes the BCG signal to obtain analysis results; the alarm logic unit is used for determining whether a human body has a vital sign signal, or an abnormal ECG signal, or an abnormal BCG signal, and for sending an alarm control signal to the alarm when the human body's vital signs signals are lost, or the ECG signal is abnormal, or the BCG signal is abnormal; the alarm emits a warning alarm after receiving the alarm control signal.

9. The ECG monitoring device of claim 8, wherein the alarm logic unit determines the abnormal ECG signal of the human body when the analysis result of the ECG signal analysis unit is abnormal and the analysis result of the BCG signal analysis unit is normal; when the analysis result of the ECG signal analysis unit is normal and the analysis result of the BCG signal analysis unit is abnormal the alarm logic unit determines that the BCG signal of the human body is abnormal; when the analysis result of the ECG signal analysis unit and the analysis result of the BCG signal analysis unit are both abnormal, the alarm logic unit determines that the vital sign signal of the human body is lost.

10. The ECG monitoring device of claim 7, wherein the ECG signal collecting electrode collects ECG signal in the manner of ECG leads.
